(19) United States
(12) Patent Application Publication
(10) Pub. No.: US 2007/0184137 A1
(43) Pub. Date: Aug. 9, 2007

(54) Morinda Citrifolia L. Based Formulations for Inhibiting Matrix Metalloproteinase Enzymes

(76) Inventors: Afa Kehaati Palu, American Fork, UT (US); Johannes Westendorf, Bremen (DE); Brett J. West, Urem, UT (US); Bing-Nan Zhou, Sandy, UT (US); Claude J. Jensen, Cedar Hills, UT (US); Stephen Story, Alpine, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/564,166

(22) Filed: Nov. 28, 2006

Related U.S. Application Data
(60) Provisional application No. 60/740,419, filed on Nov. 29, 2005.

(51) Int. Cl. A61K 36/746

(52) U.S. Cl. 424/769; 424/777

(57) ABSTRACT

The present invention relates to methods and formulations directed to the management of various Matrix Metalloproteinases enzymes comprising the administration of processed Morinda citrifolia based formulations.

### RELATED APPLICATIONS

This application claims priority to United States Provisional Application Ser. No. 60/740,419 filed Nov. 29, 2005, entitled “Morinda Citrifolia L. Based Formulations for Inhibiting Matrix Metalloproteinase”.

### BACKGROUND

1. Field of Invention

The present invention relates to methods and formulations directed to the management of Matrix Metalloproteinases enzymes comprising the administration of processed Morinda citrifolia based formulations.

2. Background

Matrix Metalloproteinases (“MMPs”) are a family of structurally related, zinc-dependent endopeptidases collectively capable of degrading components of the extracellular matrix (ECM). MMPs play a role in physiological ECM remodeling. For example MMPs are involved during tissue morphogenesis, growth, uterine cycling and postpartum involution, tissue repair, and angiogenesis. In addition, MMPs play a role in pathological conditions with excessive degradation of ECM, such as rheumatoid arthritis, osteoarthritis, atherosclerotic plaque rupture, aortic aneurysms, periodontitis, autoimmune blistering disorders of the skin, dermal photoaging, tumor invasion, and tumor metastasis.

Twenty-one human MMPs are known, at this time, and they can be divided into subgroups based on their structure and substrate specificity. These subgroups include collagenases, stromelysins and stromelysin-like MMPs, matrilysins, gelatinases, MMP19-like MMPs, membrane-type MMPs (MTMMPs), and other MMPs.

### SUMMARY AND OBJECTS OF THE INVENTION

Some embodiments of the present invention provide a method for and composition for inhibiting various Matrix Metalloproteinases enzymes.

Some embodiments of the present invention provide a method of treating various diseases and ailments, which comprise administering to said mammal a processed Morinda citrifolia product selected from a group consisting of: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates the inhibition of MMP-9 by a 100% ethanol extract of noni puree;

FIG. 2 illustrates the inhibition of MMP-9 by a 60% ethanol extract of noni puree;

FIG. 3 illustrates the inhibition of MMP-9 by a 100% ethanol extract of noni puree;

FIG. 4 illustrates the inhibition of MMP-9 by a 60% ethanol extract of noni puree;

FIG. 5 illustrates the inhibition of MMP-9 by captopril.

FIG. 6 illustrates inhibition of Calpain by TAHITIAN NONI® JUICE

FIG. 7 illustrates inhibition of Calpain enzymes by E-64 for the basis of comparison with the inhibition plots of TAHITIAN NONI® JUICE and other Morinda Citrifolia extracts and concentrates.

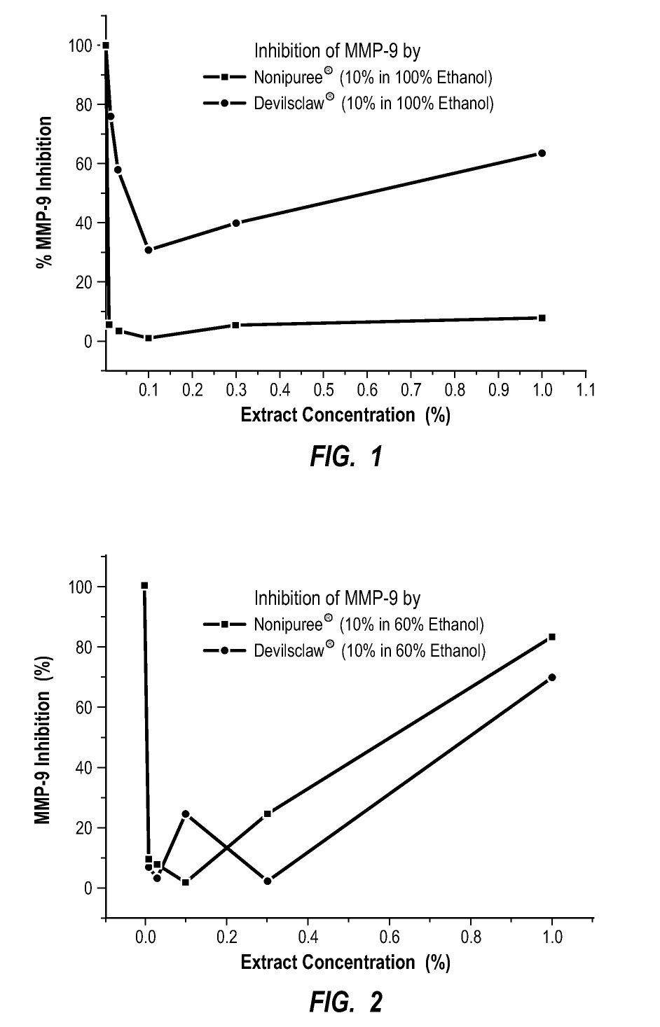

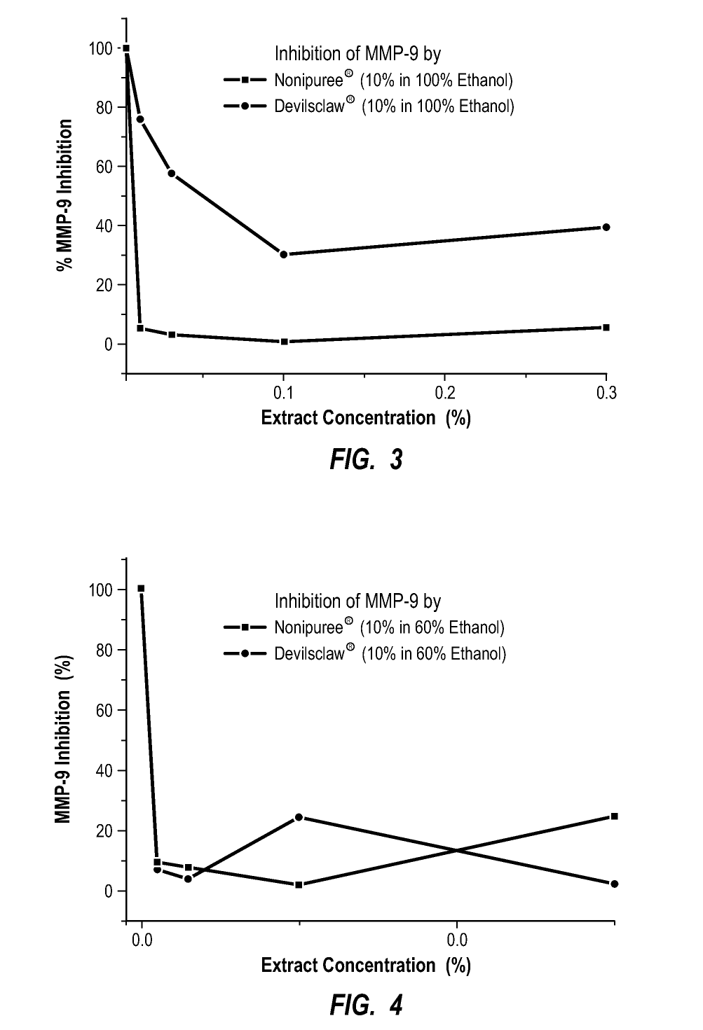

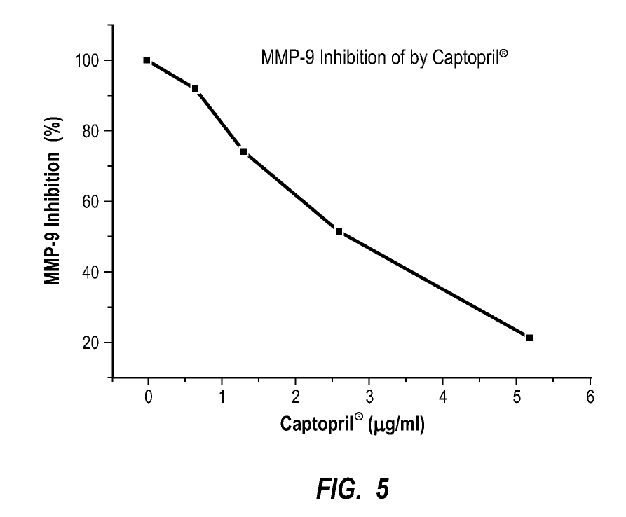

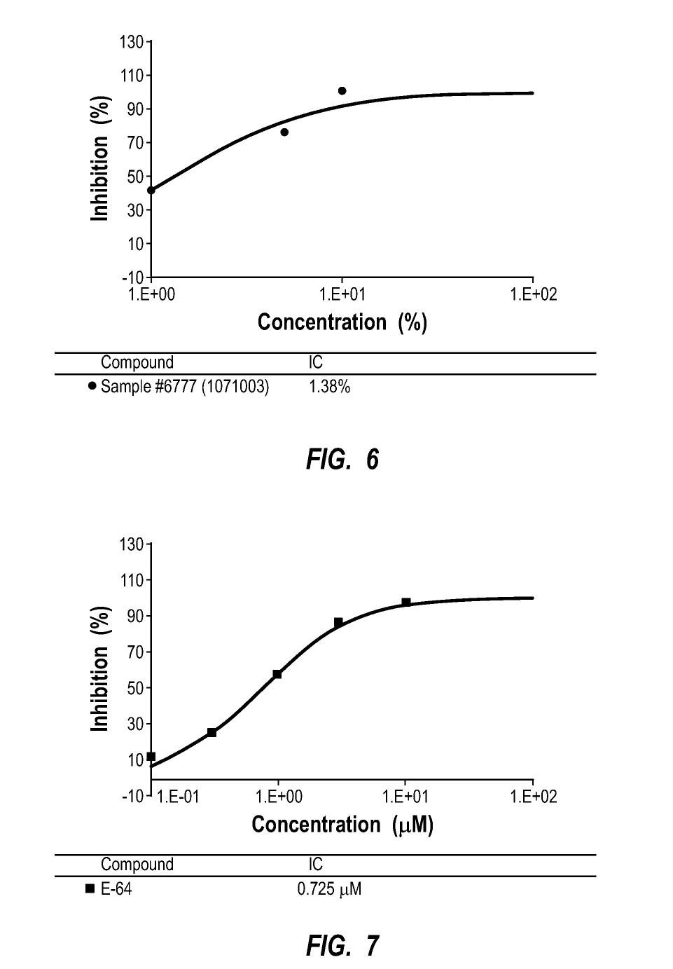

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

Embodiments of the present invention feature methods and compositions for inhibiting various Matrix Metalloproteinases enzymes and to treat and prevent pathological conditions related to tissue morphogenesis, growth, uterine cycling, postpartum involution, tissue repair, angiogenesis, excessive degradation of ECM, rheumatoid arthritis, osteoarthritis, atherosclerotic plaque rupture, aortic aneurysms, periodontitis, autoimmune blistering disorders of the skin, dermal photoaging, tumor invasion, and tumor metastasis. Moreover, embodiments of the present invention feature methods and compositions for inhibiting the growth of the second most found human skin cancer cell line. The foregoing list of ailments and diseases are mitigated, and the enzymatic inhibitions are fostered, through the administration of a composition comprising a component derived from the Indian Mulberry or Morinda citrifolia L. plant.

General Description of the Morinda citrifolia L. Plant

The Indian Mulberry or Morinda citrifolia plant, known scientifically as Morinda Citrifolia L. (“Morinda citrifolia ”), is a shrub or small tree up to 10 m in height. The leaves are oppositely arranged with an elliptic to ovate form. The small white flowers are contained in a fleshy, globose, head like cluster. The fruits are large, fleshy, and ovoid. At maturity, they are creamy white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds. When fully ripe, the fruit has a pronounced odor like rancid cheese. Although the fruit has been eaten by several nationalities as food, the most common use of the Morinda citrifolia plant has traditionally been as a red and yellow dye source.

Processing Morinda citrifolia Leaves

The leaves of the Morinda citrifolia plant are one possible component of the Morinda citrifolia plant that may be present in some compositions of the present invention. For example, some compositions comprise leaf extract and/or leaf juice as described further herein. Some compositions comprise a leaf serum that is comprised of both leaf extract and fruit juice obtained from the Morinda citrifolia plant. Some compositions of the present invention comprise leaf serum and/or various leaf extracts as incorporated into a nutraceutical product (“nutraceutical” herein referring to any drug or product designed to improve the health of living organisms such as human beings or mammals).

In some embodiments of the present invention, the Morinda citrifolia leaf extracts are obtained using the following process. First, relatively dry leaves from the Morinda citrifolia L. plant are collected, cut into small pieces, and placed into a crushing device—preferably a hydraulic press—where the leaf pieces are crushed. In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

In some embodiments of the present invention, the primary leaf extract is pasteurized to at least partially sterilize the extract and destroy objectionable organisms. The primary leaf extract is pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

In some embodiments of the present invention, the pasteurized primary leaf extract is placed into a centrifuge decanter where it is centrifuged to remove or separate any remaining leaf juice therein from other materials, including chlorophyll. Once the centrifuge cycle is completed, the leaf extract is in a relatively purified state. This purified leaf extract is then pasteurized again in a similar manner as discussed above to obtain a purified primary leaf extract.

Preferably, the primary leaf extract, whether pasteurized and/or purified, is further fractionated into two individual fractions: a dry hexane fraction, and an aqueous methanol fraction. This is accomplished preferably via a gas chromatograph containing silicon dioxide and CH2C12-MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

Processing Morinda citrifolia Fruit

Some embodiments of the present invention include a composition comprising fruit juice of the Morinda citrifolia plant. Because the Morinda citrifolia fruit is for all practical purposes inedible, the fruit must be processed in order to make it palatable for human consumption and included in the compositions of the present invention. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another product, frozen or pasteurized. In some embodiments of the present invention, the juice and pulp can be pureed into a homogenous blend to be mixed with other ingredients. Other processes include freeze drying the fruit and juice. The fruit and juice can be reconstituted during production of the final juice product. Still other processes may include air drying the fruit and juices prior to being masticated.

In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, but preferably for 2 to 3 days. The fruit is ripened or aged by being placed on equipment so that the fruit does not contact the ground. The fruit is preferably covered with a cloth or netting material during aging, but the fruit can be aged without being covered. When ready for further processing the fruit is light in color, such as a light green, light yellow, white or translucent color. The fruit is inspected for spoilage or for excessive green color and firmness. Spoiled and hard green fruit is separated from the acceptable fruit.

The ripened and aged fruit is preferably placed in plastic lined containers for further processing and transport. The containers of aged fruit can be held from 0 to 30 days, but preferably the fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.). Another product manufactured is Morinda citrifolia puree and puree juice, in either concentrate or diluted form. Puree is essentially the pulp separated from the seeds and is different than the fruit juice product described herein.

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

Processing Morinda citrifolia Seeds

Some Morinda citrifolia compositions of the present invention include seeds from the Morinda citrifolia plant. In some embodiments of the present invention, Morinda citrifolia seeds are processed by pulverizing them into a seed powder in a laboratory mill. In some embodiments, the seed powder is left untreated. In some embodiments, the seed powder is further defatted by soaking and stirring the powder in hexane—preferably for 1 hour at room temperature (Drug:Hexane—Ratio 1:10). The residue, in some embodiments, is then filtered under vacuum, defatted again (preferably for 30 minutes under the same conditions), and filtered under vacuum again. The powder may be kept overnight in a fume hood in order to remove the residual hexane.

Still further, in some embodiments of the present invention, the defatted and/or untreated powder is extracted, preferably with ethanol 50% (m/m) for 24 hours at room temperature at a drug solvent ratio of 1:2.

Processing Morinda citrifolia Oil

Some embodiments of the present invention may comprise oil extracted from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

Compositions and Their Use

The present invention features compositions and methods for inhibiting MMPs enzymes comprising the administration of processed Morinda citrifolia based formulations.

The present invention also features compositions and methods for: ameliorating pathological conditions associated with MMPs role in components of the extracellular matrix (ECM) including conditions associated with MMPs role in physiological ECM remodeling, for example during tissue morphogenesis, growth, uterine cycling and postpartum involution, tissue repair, and angiogenesis. Some embodiments relate to ameliorating pathological conditions associated with MMPs role in excessive degradation of ECM, such as rheumatoid arthritis, osteoarthritis, atherosclerotic plaque rupture, aortic aneurysms, periodontitis, autoimmune blistering disorders of the skin, dermal photoaging, tumor invasion, and tumor metastasis.

Embodiments of the present invention also comprise methods for internally introducing a Morinda citrifolia composition into the body of a mammal. Several embodiments of the Morinda citrifolia compositions comprise various different ingredients, each embodiment comprising one or more forms of a processed Morinda citrifolia component as taught and explained herein.

Compositions of the present invention may comprise any of a number of Morinda citrifolia components such as: extract from the leaves of Morinda citrifolia , leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, Morinda citrifolia seeds, Morinda citrifolia seed extracts, extracts taken from defatted Morinda citrifolia seeds, and evaporated concentration of Morinda citrifolia fruit juice. Compositions of the present invention may also include various other ingredients. Examples of other ingredients include, but are not limited to: artificial flavoring, other natural juices or juice concentrates such as a natural grape juice concentrate or a natural blueberry juice concentrate; carrier ingredients; and others as will be further explained herein.

Any compositions having the leaf extract from the Morinda citrifolia leaves, may comprise one or more of the following: the primary leaf extract, the hexane fraction, methanol fraction, the secondary hexane and methanol fractions, the leaf serum, or the nutraceutical leaf product.

In some embodiments of the present invention, active ingredients or compounds of Morinda citrifolia components may be extracted out using various procedures and processes commonly known in the art. For instance, the active ingredients may be isolated and extracted out using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health- benefiting functions just mentioned.

A non-limit example of active ingredients may include Quercetin and/or Rutin. In some embodiments such active ingredients may be present in an amount between about 0.01 and 10% by weight. In other embodiments, such active ingredients may be present in an amount between about 0.1 and 25% by weight.

Any components and compositions of Morinda citrifolia may be further incorporated into a nutraceutical product (again, “nutraceutical” herein referring to any drug or product designed to improve the health of living organisms such as human beings or mammals). Examples of nutraceutical products may include, but are not limited to: intravenous products, topical dermal products, wound healing products, skin care products, hair care products, beauty and cosmetic products (e.g., makeup, lotions, etc.), burn healing and treatment products, first-aid products, antibacterial products, lip balms and ointments, bone healing and treatment products, meat tenderizing products, anti-inflammatory products, eye drops, deodorants, antifungal products, arthritis treatment products, muscle relaxers, toothpaste, and various nutraceutical and other products as may be further discussed herein.

The compositions of the present invention may be formulated into any of a variety of embodiments, including oral compositions, topical dermal solutions, intravenous solutions, and other products or compositions.

Oral compositions may take the form of, for example, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia components in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain the Morinda citrifolia components in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweetening agents may include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. The concentration of sweeteners may be between from 0 to 50 percent by weight of the Morinda citrifolia composition, and more preferably between about 1 and 5 percent by weight.

Typical flavoring agents can include, but are not limited to, artificial and/or natural flavoring ingredients that contribute to palatability. The concentration of flavors may range, for example, from 0 to 15 percent by weight of the Morinda citrifolia composition. Coloring agents may include food-grade artificial or natural coloring agents having a concentration ranging from 0 to 10 percent by weight of the Morinda citrifolia composition.

Typical nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B12, C, D, E, Folic Acid, Pantothenic Acid, Biotin, etc. Examples of minerals and trace elements include, but are not limited to, calcium, chromium, copper, cobalt, boron, magnesium, iron, selenium, manganese, molybdenum, potassium, iodine, zinc, phosphorus, etc. Herbs and botanical extracts may include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The ingredients to be utilized in a topical dermal product may include any that are safe for internalizing into the body of a mammal and may exist in various forms, such as gels, lotions, creams, ointments, etc., each comprising one or more carrier agents. The ingredients or carrier agents incorporated into systemically (e.g., intravenously) administered compositions may also comprise any known in the art.

In one exemplary embodiment, a Morinda citrifolia composition of the present invention comprises one or more of a processed Morinda citrifolia component present in an amount by weight between about 0.01 and 100 percent by weight, and preferably between 0.01 and 95 percent by weight. Several embodiments of formulations are included in U.S. Pat. No. 6,214,351, issued on Apr. 10, 2001. However, these compositions are only intended to be exemplary, as one ordinarily skilled in the art will recognize other formulations or compositions comprising the processed Morinda citrifolia product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0. 1-20 percent; and a carrier medium present in an amount by weight between about 20-90 percent. Morinda citrifolia puree juice or fruit juice may also be formulated with a processed Morinda citrifolia dietary fiber product present in similar concentrations.

### EXAMPLES

The following examples illustrate some of the embodiments of the present invention comprising the administration of a composition comprising components of the Indian Mulberry or Morinda citrifolia L. plant. These examples are not intended to be limiting in any way, but are merely illustrative of benefits, advantages, and remedial effects of some embodiments of the Morinda citrifolia compositions of the present invention.

As illustrated by the following Examples, embodiments of the present invention have been tested against various Matrix Metalloproteinases enzymes. Specifically, the Example illustrate the results of in-vitro studies that confirmed that concentrates of processed Morinda citrifolia products wherein “TNJ” is an evaporative concentrate, “TNCONC” is a freeze concentrate, Noni Puree is a Morinda citrifolia based puree produced as described in this invention, Compound 1 are Noni concentrates and Noni leaf active fractions could have productive affects on various MMPs in-vivo. The percentage of concentration refers to the concentration strength of the particular concentrate tested; that is, the strength of concentration relative to the processed Morinda citrifolia product from which the concentrate was obtained.

All MMP inhibition assays discussed in Examples 1-4 were conducted utilizing the protocol outlined in Table 1 below:

114310 Peptidase, Matrix Metalloproteinase-3 (MMP-3)

| | |
|---|---|
| Source: | Human recombinant |
| Substrate: | 4 μM Mca-Pro-Leu-Gly-Leu-Dpa-Ala-Arg- |
| | NH₂ |
| Vehicle: | 1% DMSO |
| Pre-Incubation Time/Temp: | 60 minutes @ 37° C. |
| Incubation Time/Temp: | 2 hours @ 37° C. |
| Incubation Buffer: | 50 mM MOPS, 10 mM CaCl₂2H₂O, 10 μM |
| | ZnCl₂, 0.05% Brij 35, pH 7.2 |
| Quantitation Method: | Soectrofluorimetric quantitationof Mca- |
| | Pro-Leu-Gly-NH₂ |
| Significance Criteria: | ≧50% of max stimulation or inhibition |

### Example 1

Results for Morinda citrifolia Based Freeze Concentrates

As shown in the following plot Morinda citrifolia freeze concentrates potent inhibitors of the metalloproteinase MMP-1, MMP-3 and MMP-9.

| | | Sample | | Percent |
|---|---|---|---|---|
| Enzyme | Source | Size | Concentration | Inhibition |
| Peptidase, Matrix | | | | |
| Metalloproteinase- | Hum | 2 | 10% | 87 |
| 1 (MMP-1) | | | | |
| Metalloproteinase- | Hum | 2 | 10% | 85 |
| 3 (MMP-3) | | | | |
| Metalloproteinase- | Hum | 2 | 10% | 72 |
| 9 (MMP-9) | | | | |

### Example 2

Results for Noni Leaves Active Fractions

As shown in the following plot noni leaf active fractions are potent inhibitors of the metalloproteinase MMP-1, MMP-2, MMP-3 and MMP-9.

| | | | | |
|---|---|---|---|---|
| | | | Percent | |
| Enzyme | Source | Concentration | Inhibition | IC₅₀ |

Peptidase, Matrix Metalloproteinase-1 (MMP-1)

| | | | | | |
|---|---|---|---|---|---|
| TNL₃ | 1060522 | Hum | 1% | 76 | 0.517% |

Peptidase, Matrix Metalloproteinase-2 (MMP-2)

| | | | | | |
|---|---|---|---|---|---|
| TNL₃ | 1060522 | Hum | 0.5% | 61 | 0.234% |

Peptidase, Matrix Metalloproteinase-3 (MMP-3)

| | | | | | |
|---|---|---|---|---|---|
| TNL₃ | 1060522 | Hum | 0.5% | 67 | 0.184% |

Peptidase, Matrix Metalloproteinase-9 (MMP-9)

| | | | | | |
|---|---|---|---|---|---|
| TNL₃ | 1060522 | Hum | 0.5% | 58 | 0.302% |

### Example 3

Results for Tahitian Noni Juice ® Effects on MMPs

As shown in the following plot TAHITIAN NONI® JUICE is a potent inhibitor of the metalloproteinase MMP-7 and MMP-13.

| | | Sample | | Percent |
|---|---|---|---|---|
| Enzyme | Source | Size | Concentration | Inhibition |
| Peptidase, Matrix | | | | |
| Metalloproteinase-7 | Hum | 2 | 10% | 87 |
| (MMP-7) | | | | |
| Metalloproteinase-13 | Hum | 2 | 10% | 87 |
| (MMP-13) | | | | |
| Peptidase, | | | | |
| Metalloproteinase | | | | |
| Neutral Endopeptidase | Hum | 2 | 10% | 57 |
| | | 2 | 5% | 46 |
| | | 2 | 1% | 45 |

### Example 4

Results for both TNJ and TNCMP1

For the purposes of the following assay Sample #100 refers to TAHITIAN NONI JUICE® and TNCMP1 refers to Noni Concentrate (Morinda Compound 1). As shown both TAHITIAN NONI JUICE® and Noni Concentrate are potent inhibitors of MMP-12.

| | | | | |
|---|---|---|---|---|
| | | Sample | | Percent |
| Sample | Source | Size | Concentration | Inhibition |

115200 Peptidase, Matrix, Metalloproteinase-12 (MMP-12)

| | | | | |
|---|---|---|---|---|
| Sample | Hum | 2 | 5% | 93 |
| #100 | | | | |
| | | 2 | 1% | 78 |
| TNCMP1 | Hum | 2 | 5% | 99 |
| | | 2 | 1% | 102 |

### Example 5

As shown in the following plot TAHITIAN NONI JUICE ® is a potent inhibitor of Calpain-1, which is involved in various neurodegenerative disorders.

| TARGET | SPP. | N = | Conc. | % Inhibition | IC₅₀ |
|---|---|---|---|---|---|
| Peptidase, | Hum | 2 | 10% | 101 | 1.38% |
| Calpain-1 | | | | | |
| | | 2 | 5% | 76 | |
| | | 2 | 1% | 41 | |

The following table provides the methods and materials utilized to conduct the assay described in Example 5.

108010 Peptidase, Calpain-1

| | |
|---|---|
| Source: | Human erythrocytes |
| Substrate: | 0.05% Casein-FITC |
| Vehicle: | 1% DMSO |
| Pre-Incubation Time/Temp: | Non |
| Incubation Time/Temp: | 30 minutes @ 37° C. |
| Incubation Buffer: | 50 mM Tris-HCl, pH 7.4 |
| Quantitation Method: | Spectrofluorimetric quantitation of Pettide- |
| | FITC |
| Significance Criteria: | ≧50% of max stimulation or inhibition |

### Example 6

For the purpose of the following assay TNCMP 1 refers to Noni Concentrate. As shown in the following plot Noni Concentrate is a potent inhibitor of Calpain, which is involved in various neurodegenerative disorders.

| Peptidase, | | Sample | | | |
|---|---|---|---|---|---|
| Calpain-1 | Source | Size | Concentration | % Inhibition | IC₅₀ |
| TNCMP1 | Hum | 2 | 10% | 90 | <1% |
| | | 2 | 5% | 96 | |
| | | 2 | 1% | 69 | |

The present invention may be embodied in other specific forms without departing from its spirit of essential characteristics. The described embodiments are to be considered in all respects only as illustrative and not restrictive. The scope of the invention is, therefore, indicated by the appended claims, rather than by the foregoing description. All changes that come within the meaning and range of equivalency of the claims are to be embraced within their scope.

What is claimed is:

1. A formulation adapted for inhibiting various metalloproteinase in mammals comprising: a processed Morinda citrifolia product.

2. The formulation of claim 1, wherein said Morinda citrifolia product is used with a carrier medium.

3. The formulation of claim 1, wherein said processed Morinda citrifolia product comprises a processed Morinda citrifolia selected from a group consisting of: extract from the leaves of Morinda citrifolia , leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

4. The formulation of claim 1, comprising at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

5. The formulation of claim 1, wherein said formulation is administered to a patient by at least one method selected from a list consisting of orally, intravenously, and systemically.

6. The formulation of claim 1, further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

7. A method for inhibiting metalloproteinase activity profiles in mammals comprising the steps of:
administering a formulation comprising a Morinda citrifolia product;
inhibiting a metalloproteinase enzyme in said animal.

8. The method of claim 7, wherein two ounces of the formulation is administered twice daily.

9. The method of claim 7, wherein said Morinda citrifolia product is administered with a carrier medium.

10. The method of claim 7, wherein said processed Morinda citrifolia product comprises a processed Morinda citrifolia selected from a group consisting of: extract from the leaves of Morinda citrifolia , leaf hot water extract present in an amount by weight between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

11. The method of claim 7, wherein the formulation comprises at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

12. The method of claim 7, wherein the formulation further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

13. The method of claim 7, wherein said formulation is administered in an amount between about 1 teaspoon and 2 ounces at least twice daily on an empty stomach each day.
